(19) United States
(12) Patent Application Publication
(10) Pub. No.: US 2025/0027174 A1
(43) Pub. Date: Jan. 23, 2025

(54) SYSTEMS INCLUDING NUCLEIC ACID DETECTION DEVICES AND RELATED METHODS THEREOF

(71) Applicant: Nur Biosolutions, LLC, Germantown, MD (US)

(72) Inventor: Raza ZAIDI, Germantown, MD (US)

(21) Appl. No.: 18/778,529

(22) Filed: Jul. 19, 2024

Related U.S. Application Data
(60) Provisional application No. 63/514,944, filed on Jul. 21, 2023.

(51) Int. Cl. C12Q 1/70; B01L 3/00; B01L 7/00; C12Q 1/6844; G01N 27/04

(52) U.S. Cl. CPC C12Q 1/701; B01L 3/502715; B01L 7/52; C12Q 1/6844; G01N 27/04; B01L 2200/16; B01L 2300/027; B01L 2300/0636; B01L 2300/0861; B01L 2300/18

(57) ABSTRACT

The teachings generally provide for a diagnostic device, including a sample chamber configured to receive a sample from a patient, a plurality of reaction chambers, a heating element in thermal communication with the plurality of reaction chambers, a plurality of probes extending from each of the reaction chambers to a data processor. The reaction chambers are configured to facilitate a loop-mediated isothermal amplification (LAMP) test. The plurality of reaction chambers include a sample reaction chamber and a control chamber, the sample reaction chamber configured to receive the sample and the control chamber is configured to be isolated from the sample. The plurality of probes communicates with the data processor to provide test data from the sample reaction chamber and the control reaction chamber so that the data processor generates test results based on a comparison of the test data from the sample reaction chamber and the control reaction chamber.

### CROSS-REFERENCE TO RELATED APPLICATION

This application claims priority to U.S. Provisional Application No. 63/514,944, filed on Jul. 21, 2023, the entirety of which is incorporated by reference herein.

### SEQUENCE LISTING

This application contains a sequence listing, submitted electronically in XML format under the filename 00390-0001-01000.xml, which is incorporated by reference herein in its entirety. The XML copy of the sequence listing was created on Jul. 19, 2024, and is 142,362 bytes in size.

### TECHNICAL FIELD

Various aspects of this disclosure relate generally to systems including nucleic acid detection devices and related methods. For example, systems and methods of the present disclosure may be used to detect the presence of one or more nucleic acid sequences in a sample.

### INTRODUCTION

Early detection of an infection is often helpful in the treatment of infectious diseases and the spread of infectious diseases. Accordingly, tests that are fast, affordable, and accurate may be desirable. Such rapid tests may enable subjects who believe they may have been exposed to a particular disease to quickly and easily receive test results that may inform treatment and/or containment procedures. Conventional devices used to detect the presence of common infections, such as polymerase chain reaction (PCR)-based tests, require laboratory-based processing of samples. Therefore, a significant amount of time, sometimes up to several days, may be necessary to process a sample and determine a result. Other conventional devices used to diagnosis infectious diseases may be have high associated costs, high error-rates, and/or are limited to the detection of one specific type of infection.

The introduction is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

This Summary introduces a selection of concepts in a simplified form that are further described below in the Detailed Description below. This Summary is not intended to limit the scope of the claimed subject matter nor identify key features or essential features of the claimed subject matter.

The teachings generally provide for a diagnostic device, including a sample chamber configured to receive a sample from a patient, a plurality of reaction chambers, a heating element in thermal communication with the plurality of reaction chambers, a plurality of probes extending from each of the reaction chambers to a data processor. The reaction chambers are configured to facilitate a loop-mediated isothermal amplification (LAMP) test. The plurality of reaction chambers include a sample reaction chamber and a control chamber, the sample reaction chamber configured to receive the sample and the control chamber is configured to be isolated from the sample. The plurality of probes communicates with the data processor to provide test data from the sample reaction chamber and the control reaction chamber so that the data processor generates test results based on a comparison of the test data from the sample reaction chamber and the control reaction chamber.

### BRIEF DESCRIPTION OF THE DRAWINGS

The accompanying drawings, which are incorporated in and constitute a part of this specification, illustrate aspects of this disclosure and together with the description, serve to explain the principles of the disclosure. Moreover, the described systems and methods are neither limited to any single aspect nor embodiment thereof, nor to any combinations or permutations of such aspects and embodiments. For the sake of brevity, certain permutations and combinations are not discussed and/or illustrated separately herein.

FIG. 1A shows a schematic view of a testing device, according to embodiments of the present disclosure.

FIG. 1B shows a schematic view of a testing device, according to embodiments of the present disclosure.

FIG. 1C shows a schematic view of a testing device, according to embodiments of the present disclosure.

FIGS. 2A and 2B illustrate one example of a locking mechanism, according to embodiments of the present disclosure.

FIG. 3 illustrates an exemplary LAMP based nucleic acid amplification, according to embodiments of the present disclosure.

FIG. 4 depicts, in flowchart form, an exemplary method of using a testing device, according to embodiments of the present disclosure.

FIG. 5 is an example diagram of a computing device, according to embodiments of the present disclosure.

### DETAILED DESCRIPTION

It may be understood that both the foregoing general description and the following detailed description are exemplary and explanatory only and are not restrictive of the invention, as claimed. As used herein, the terms “comprises,” “comprising,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “top” refers to a direction or side of a device relative to its orientation during use, and the term “bottom” refers to a direction or side of a device relative to its orientation during use that is opposite of the “top.” The term “distal” refers to a direction away from an operator/toward a treatment site, and the term “proximal” refers to a direction toward an operator. The term “exemplary” is used in the sense of “example,” rather than “ideal.”

As used herein, the term “approximately” is meant to account for variations due to experimental error. When applied to numeric values, the term “approximately” may indicate a variation of +/−5% from the disclosed numeric value, unless a different variation is specified. As used herein, the singular forms “a,” “an,” and “the” include plural referents unless the context clearly dictates otherwise. Further, all ranges are understood to be inclusive of endpoints, e.g., from 1 mm to 5 mm would include 1 mm and 5 mm and all distances or lengths between 1 mm and 5 mm.

To provide testing accessibility to subjects, at-home test kits may be a practical alternative to testing conducted in a doctor's office or health clinic. Subjects may avoid waiting for an appointment with a medical professional, and may conduct tests themselves at their convenience, within their own homes. In addition to the flexibility and convenience provided by at-home test kits, the option to avoid in-office testing may further help reduce the spread of infectious diseases, as infected subjects may limit their contact with other subjects who may be visiting the doctor or clinic.

Some conventional at-home test kits may allow for the detection of a single infectious agent. However, there exists a need for test devices capable of detecting multiple infections, such as, for example, simultaneously testing for the presence of multiple infection agents.

Systems and methods of the present disclosure may allow for the detection of one or more infectious agents in a sample. For example, systems and methods of the present disclosure may allow for the detection of the presence one or more infectious diseases indicators in a sample. Systems and methods of the present disclosure may allow for the detection of one or more target sequences that are unique to a particular infectious agent.

Systems and methods of the present disclosure may include amplifying molecules of an analyte (e.g., a sample from a subject) that match a target nucleic acid sequence. Systems of the present disclosure may comprise a testing device including one or more reaction chambers. Each reaction chamber may comprise the necessary reagents (e.g., buffers, primers, enzymes, polymerases, nucleotides) for a nucleic acid amplification process and may be configured to receive a sample. If the sample includes molecules that match a target nucleic acid sequence, those target sequences of the sample molecules may be amplified. The presence of intermediates, byproducts, or other products of the amplification process may be used to detect the presence of molecules in the sample that match a target nucleic acid sequence. In some embodiments, other measureable properties of the amplification process (e.g., a pH, a conductivity, a temperature, a viscosity, a transmittance, an absorbance, or a combination thereof) may be used to detect the presence of molecules in the sample that match a target nucleic acid sequence.

Systems of the present disclosure may be compatible with multiple types of samples. For example, systems of the present disclosure may analyze samples in the form of bodily fluids (e.g., mucus, saliva, blood, tears), epithelial swabs, or other sample including living cells from a subject.

In some examples, collection of a patient sample and preparation may include acquisition of a sample from a patient with a sterile swab. The swab may then be rinsed in a pre-filled extraction tube to remove at least a portion of the sample from the swab into the extraction tube. The extraction tube may be filled with reaction solution. The reaction solution and at least a portion of the sample may be loaded into a testing device, as described herein.

As alluded to above, systems of the present disclosure may include a testing device. The testing device may be configured as a single-use or a combination multiplex diagnostic system utilizing nucleic acid amplification for the detection of infectious diseases. The diagnostic system may be utilized by subjects in an at-home setting. The diagnostic system may be used in a variety of environments, such as, for example, locations where a laboratory testing environment is not accessible and that would benefit from the availability of infectious diseases diagnoses. Some non-limiting examples where the diagnostic system may be used may include airports, cruise ships, large venues, office buildings, homes, and/or other residential or mass occupancy enclosures. Accordingly, in some examples, a subject may conduct a test utilizing the diagnostic system as a form of screening for a disease before entering a venue hosting a large group of people. In addition or alternatively, a subject may conduct a test as soon as the subject presents symptoms, or becomes aware of potential exposure to a disease, and may avoid a visit to a doctor or a medical clinic. Embodiments of the present disclosure may be utilized to quickly and conveniently obtain test results, while limiting potential exposure of uninfected subjects.

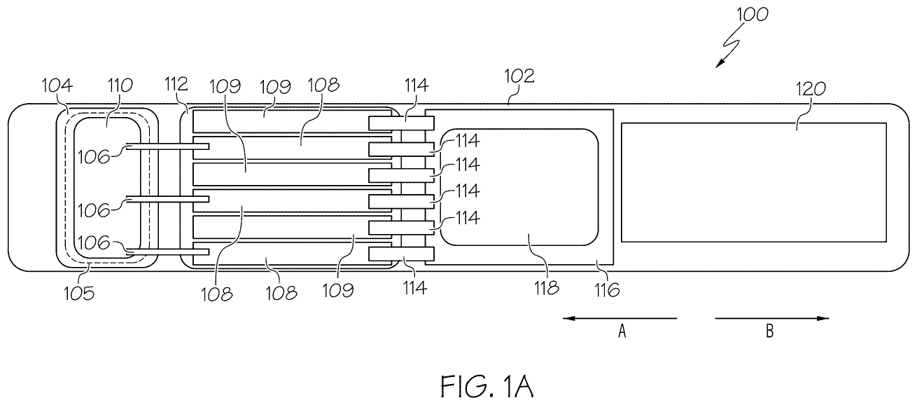

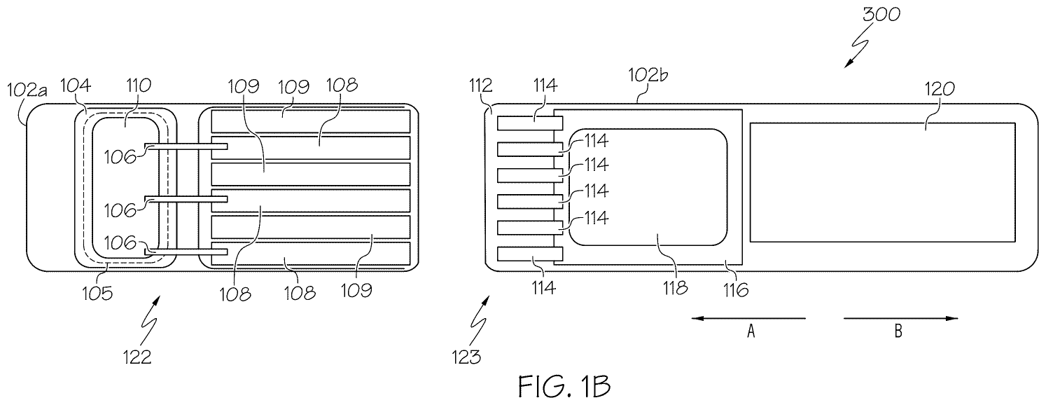

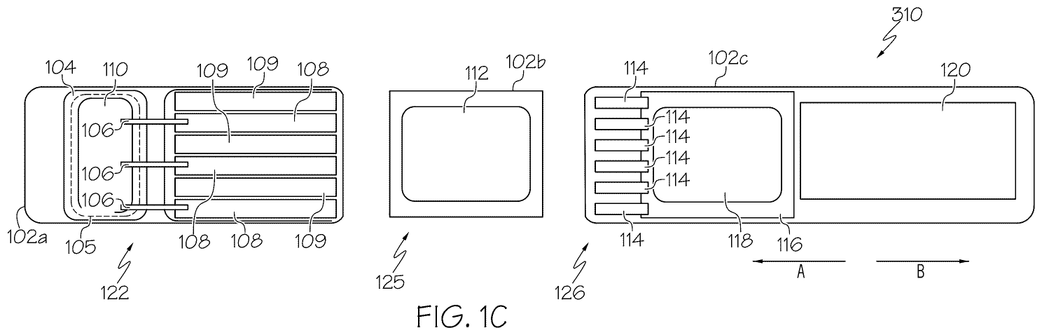

The diagnostic system may include a testing device 100. Referring to FIG. 1A, an exemplary testing device 100 is shown. Testing device 100 may include a housing 102. The housing 102 may contain other components of testing device 100, such as, for example, one or more sample chambers 104, distributors 110, channels 106, sample reaction chambers 108, control reaction chambers 109, heating element 112, probes 114, circuit boards 116, data processors 118 and/or displays 120. In some examples, control reaction chambers 109 may be configured to be isolated from the sample. In some examples, heating element 112 may be configured to surround or substantially surround control reaction chambers 109.

Sample chamber 104 may be configured to receive a sample (e.g., a reaction solution from an extraction tube). The sample chamber 104 may be sized and shaped to receive a suitable sample size from a subject, depending on the type of sample being tested. In some embodiments, sample chamber 104 may have a volume of approximately 1 μL to approximately 150 μL, such as, for example, a volume of approximately 50 μL or less, a volume of approximately 10 μL or less, or a volume of approximately 2 μL to approximately 35 μL. In some embodiments, sample chamber 104 may be made from a polymeric material (e.g., polypropylene), or any other material suitable for use in a medical device.

In some examples, sample chamber 104 may include a sealing mechanism (not shown), which may be activated by closing of the sample chamber 104 with a cover 105. The sealing mechanism may comprise a portion of the sample chamber 104, a cover 105, and a seal configured to be disposed between the sample chamber 104 and the cover 105, when the cover 105 is placed over the sample chamber 104. In some examples, the sealing mechanism may be connected with the sample chamber 104, the housing 102 of the testing device 100, or both. In another example, at least a portion of the sealing mechanism may be removably connected with the sample chamber 104, the housing 102, or both. The sealing mechanism may be a one-way sealing mechanism, which may prevent a user from opening sample chamber 104 once it has been connected with the sample chamber 104, for example by closing a cover of sample chamber 104. Some examples of a sealing mechanism may be a cover, a door, a sliding member, a cap, or any other covering or closure that is configured to seal the sample chamber 104.

A testing device (e.g., testing device 300 or testing device 310) may include two or three separate portions. One or more of the separate portions may be reusable. For example, a first portion may be designed to removably coupled to the second portion. In some embodiments, a first portion of testing device 300 is a single use portion and a second portion of testing device 300 is reusable. Components that are sensitive to potential contamination, require active reagents, and/or must be kept sterile before use may be included in the single use portion. Components that are costly, complex, and/or not sensitive to potential contamination may be included in the reusable portion.

Referring to FIG. 1B, a testing device 300 may include a first portion 122 and a second portion 123. First portion 122 may include sample chamber 104, cover 105, distributors 110, channels 106, sample reaction chambers 108, and/or control reaction chambers 109. Components of first portion 122 may be positioned within a housing 102a. Second portion 123 may include heating element 112, probes 114, circuit board 116, data processor 118, and/or display 120. Components of second portion 123 may be positioned within a housing 102b.

Each of first portion 122 and second portion 123 may be single-use or reusable. For example, first portion 122 may be single-use and second portion 123 may be reusable. A first sample may be loaded into sample chamber 104 of first portion 122. Before or after the sample is loaded into first portion 122, first portion 122 may be interfaced with second portion 123. After results of the testing device 300 are displayed on display 120, first portion 122 may be removed from second portion 123 and discarded. Optionally, second portion 123 may then be interfaced to another first portion 122 (e.g., a first portion 122 that received a second sample.)

Referring to FIG. 1C, a testing device 310 may include a first portion 122, a heating portion 125, and an analysis portion 126. First portion 122 may include sample chamber 104, cover 105, distributors 110, channels 106, sample reaction chambers 108, and/or control reaction chambers 109. Components of first portion 122 may be positioned within a housing 102a. Heating portion 125 may include a heating element 112 disposed within a housing 102b. Analysis portion 126 may include probes 114, circuit board 116, data processor 118, and/or display 120. Components of analysis portion 126 may be positioned within a housing 102c.

Each of first portion 122, heating portion 125, and analysis portion 126 may be single-use or reusable. For example, first portion 122 may be single-use, heating portion 125 may be reusable, and analysis portion 126 may be reusable. A first sample may be loaded into sample chamber 104 of first portion 122. Before or after the sample is loaded into first portion 122, first portion 122 may be interfaced with heating portion 125. After an indication from heating portion 125 or first portion 122, the first portion 122 may be disconnected from heating portion 125. After first portion 122 is disconnected from heating portion 125, the first portion 122 may be interfaced with analysis portion 126. After results of the testing device 310 are displayed on display 120, first portion 122 may be removed from analysis portion 126 and discarded. Optionally, heating portion 125 and/or analysis portion 126 may then be interfaced to another first portion 122 (e.g., a first portion 122 that received a second sample.)

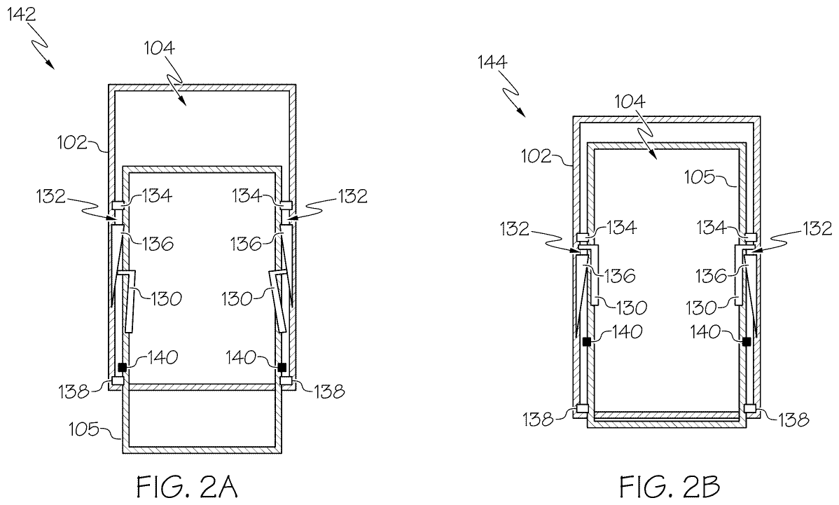

FIGS. 2A and 2B illustrate one example of the sealing mechanism. The sealing mechanism may include cover 105 configured to be slid along housing 102 from an open position 142 to a closed position 144. In open position 142, cover 105 does not extend over sample chamber 104. In closed position 144, cover 105 extends over sample chamber 104 initiating the testing.

To maintain open position 142 and closed position 144 of sealing mechanism, housing 102 may include stops 138 at a distal end of housing 102. Cover 105 may similarly include protrusions 140 to interface with stops 138. Stops 138 may be configured to stop cover 105 from being removed from housing 102 inadvertently (e.g., via the interaction of stops 138 with protrusions 140).

The sealing mechanism may include features on housing 102 and cover 105 that may lock and/or seal cover 105 to housing 102. Cover 105 may include locking arms 130. Locking arms 130 may be configured to assist with sealing cover 105 to housing 102 such that a user may not re-open cover 105 after it has been closed. In some examples, locking arms 130 may be an arm with a protrusion extending from the arm, such as shown in FIGS. 2A and 2B. Locking arms 130 may be configured to pivot and/or bias relative to cover 105 and engage one or more receiving features of housing 102 to seal cover 105 to housing 102. Locking arm 130 may be configured to bias against portions of housing 102 so that when cover 105 is moved into closed position 144, locking arms 130 may be forced into the receiving features (e.g., receiver 132) on housing 102. Although the present example shows two locking arms 130, a single locking arm 130 may be used. In other examples, any number of locking arms may be used.

The sealing mechanism may include receiving features (e.g., receiver 132) on housing 102. Receiver 132 may be configured to receive locking arms 130 from cover 105. Receiver 132 may be a portion of housing 102, may be one or more features added to housing 102, or both. Receiver 132 may be configured as a slot or a pocket to receive locking arms 130. In this example, receiver 132 may include forward stop 134 and ramp 136, described further below.

As indicated above, receiver 132 may include forward stop 134. Forward stop 134 may be configured to locate locking arms 130 of cover 105 in receiver 132. In this example, forward stop 134 is a protrusion projecting from housing 102. Although the present example illustrates forward stop 134 as a protrusion, any suitable shape or design may be used.

Ramp 136 may be configured to assist with locating locking arm 130 into receiver 132. Ramp 136 may be configured to apply a force onto locking arms 130, causing locking arms to bias inwards. Ramps 136 may be taper to provide this biasing force onto locking arms 130 in order to compress the protrusion of locking arms 130. By compressing locking arms 130 and the protrusion extending from locking arms 130 as the cover 105 is moved between positions, locking arms 130 will snap into receiver 132 when cover 105 is moved from open position 142 to closed position 144.

Although not shown, housing 102, cover 105, locking arms 130, receiver 132, or a combination thereof may include a contact for completing a circuit. The circuit may be connected with heating element 112, probes 114, circuit board 116, data processor 118, or a combination thereof. When the cover 105 is moved into the closed position 144, the circuit may be completed to begin the testing reactions.

Connected with sample chamber 104 is distributor 110. The distributor 110 may function to distribute the sample from the sample chamber 104 to reaction chambers 108 when the cover 105 is secured to the sample chamber 104. The distributor 110 may be configured as a manifold to distribute fluid from one location to another, such as moving the sample from the sample chamber 104 to the sample reaction chambers 108. The distributor 110 may include channels 106 to move a portion of the sample from the sample chamber 104 to the sample reaction chambers 108. In some examples, the distributor 110 may distribute approximately 2-5 μL of the sample to each of the sample reaction chambers 108. As the sealing mechanism is sealed against the housing 102, a positive pressure is applied to the sample, pushing the sample through the distributor 110 into the channels 106 toward the sample action chambers 108.

The sample chamber 104 may be in fluid communication with sample reaction chambers 108 via a plurality of channels 106. For example, each sample reaction chamber 108 may include a corresponding channel 106 that connects the sample reaction chamber 108 to the sample chamber 104. In some embodiments, sealing the sealing mechanism may advance the sample from sample chamber 104 through the distributor 110 into the sample reaction chambers 108 (e.g., via channels 106). For example, the sealing mechanism may include an actuation device configured to move the sample in a direction “B,” as indicated by the arrows shown in FIG. 1A, toward sample reaction chambers 108. In one example, the sealing mechanism may be configured as a cover 105 and configured to be received by the sample chamber 104, such that securing the cover 105 with the sample chamber 104 creates a positive pressure to push the sample from the sample chamber 104 into the sample reaction chambers 108. In some configurations, when the sealing mechanism includes a cover 105, the sealing mechanism may include a gasket or other type of deformable seal to securely couple the sealing mechanism with the sample chamber 104.

The testing device 100, 300, 310 may include one or more seals between sample chamber 104 and sample reaction chambers 108 which may prevent contaminants from entering sample reaction chamber 108, prior to the introduction of the sample. In addition or alternatively, testing device 100 may include one or more seals between sample chamber 104 and sample reaction chambers 108 that prevents the sample from moving in a direction “A” after the sample is introduced into sample reaction chambers 108. For example, each channel 106 connecting the sample chamber 104 to a sample reaction chamber 108 may include a one-way valve.

Each of the sample reaction chambers 108 may have a volume sufficient to perform a nucleic acid amplification reaction using the sample as an analyte. The exemplary testing device 100 in FIG. 1A includes three sample reaction chambers 108 and three control reaction chambers 109, however, it is contemplated that any number of reaction chambers 108, 109 may be incorporated, depending on the overall dimensions and intended functionality of testing device 100. For example, in some embodiments, there may be a 1:1 ratio of sample reaction chambers 108 to control reaction chambers 109, such that each sample reaction chamber 108 corresponds to a control reaction chamber 109. In other embodiments, there may be a higher ratio of sample reaction chambers 108 to control reaction chambers 109, such as, for example, 2:1, 3:1, or 4:1.

Testing device 100, 300, 310 may include a plurality of sample reaction chambers 108, where the volume of each sample reaction chamber 108 is the same. In some embodiments, a testing device 100, 300, 310 may include a first sample reaction chamber 108 with a larger volume than a second sample reaction chamber 108. Each sample reaction chamber 108 may independently have a volume of approximately 5 μL to approximately 100 μL, such as, for example, approximately 10 μL to approximately 50 μL, or approximately 27 μL to approximately 30 μL.

Testing device 100, 300, 310 may include a plurality of control reaction chambers 109, where the volume of each control reaction chamber 109 is the same. In some embodiments, a testing device 100, 300, 310 may include a first control reaction chamber 109 with a larger volume than a second control reaction chamber 109. Each control reaction chamber 109 may independently have a volume of approximately 5 μL to approximately 100 μL, such as, for example, approximately 10 μL to approximately 50 μL, or approximately 27 μL to approximately 30 μL.

Sample reaction chambers 108 may have a volume that is the same as the control reaction chambers 109 (e.g., each sample reaction chamber 108 has a volume that is equal to a volume of a corresponding control reaction chamber 109). In other examples, testing device 100, 300, 310 may include sample reaction chambers 108 with greater or lesser volumes than control reaction chambers 108.

Prior to the introduction of the sample into the sample reaction chambers 108, the contents of sample reaction chambers 108 may be the same as the contents of control reaction chambers 109. Each sample reaction chamber 108 and control reaction chamber 109 may include reagents for a nucleic acid amplification reaction, such as, for example, buffers, primers, enzymes, polymerases (e.g., BST polymerase, BSM polymerase), nucleotides (e.g., NTPs).

As described herein, testing device 100 may utilize nucleic acid amplification testing to determine the presence of one or more target sequences within a sample. In some examples, nucleic acid amplification may include loop-mediated isothermal amplification (LAMP). LAMP uses 4-6 primers recognizing 6-8 distinct regions of target DNA for a highly specific amplification reaction. A strand-displacing DNA polymerase initiates synthesis and two specially designed primers form “loop” structures to facilitate subsequent rounds of amplification through extension on the loops and additional annealing of primers. DNA products are very long (>20 kb) and formed from numerous repeats of the short (80-250 bp) target sequence, connected with single-stranded loop regions in long concatamers. Since DNA products are so long, they are not typically appropriate for downstream manipulation. However, target amplification of specific areas of the DNA is so extensive that numerous modes of detection are possible, such as change in pH and/or change in conductivity, as discussed further below.

Each reaction chamber 108, 109 may include a volume of reaction mixture. The reaction mixture may include buffers, primers, enzymes, polymerases (e.g., BST polymerase, BSM polymerase), nucleotides (e.g., NTPs), and/or other reagents necessary for the nucleic acid amplification. In some embodiments, the reaction mixture may include a commercially available master mix (e.g., WarmStart Colormetric LAMP 2×Master Mix with UDG available from New England Biolabs in Ipswich, Massachusetts) that includes one or more polymerases, one or more enzymes (e.g., uracil-DNA glycosylase), dNTPs, buffer salts (e.g., MgCl₂), one or more pH indicators (e.g., a visible pH indicator), and/or dUTP. A composition of one exemplary reaction mixture is summarized in Table 1, with amounts shown as a volume percentage (vol. %), based on the total volume of the reaction mixture. The master mix described in Table 1 is WarmStart Colormetric LAMP 2×Master Mix with UDG, comprising WarmStart DNA Polymerase BST2.0, dNTPs, MgCl2, a visible pH indicator, dUTP, and uracil-DNA glycosylase.

TABLE 1

| Reagent | Amount (vol. %) |
|---|---|
| Master Mix | 50% |
| Primer Mix | 10% |
| Nuclease-free Water | 40% |

Those of ordinary skill will understand that the volume percentages included in Table 1 are one example, and other ratios of reagents may be included in the reaction mixtures described herein. For example, the exact reaction mixtures and primer compositions may depend on the target sequence being tested, the type of sample used, and/or the type of nucleic acid amplification utilized by the testing device 100, 300, 310. As described herein, the sample may be introduced into reaction chambers 108 (e.g., via distributor 110) and added to reaction mixture. The sample may be combined with the reaction mixture at a volume ratio of approximately 1:20 to approximately 1:30, such as, for example, 1:25.

In a multiplex testing device 100, 300, 310, a first sample reaction chamber 108 may include different primers than the other sample reaction chambers 108 of the testing device 100, 300, 310. In addition or alternatively, a first control reaction chamber 109 of the multiplex testing device 100, 300, 310 may include different primers than other control reaction chambers 109 of the testing device 100, 300, 310. Target sequences being tested may include those that are specific to an infectious agent. For example, testing device 100, 300, 310 may be configured to detect target sequences from influenza (e.g., H1N1), SARS-COV-2 (coronavirus), respiratory syncytial virus (RSV), Middle Eastern Respiratory Syndrome (MERS), measles, canine distemper virus (CDV), Canine parvovirus type 2 (CPV2), canine adenovirus 1 (CAV-1), feline calcivirus (FCV), feline immunodeficiency virus (FIV), felid alphaherpesvirus (FeHV-1), cucumber mosaic virus (CMV), tabacco mosaic virus (TMV), tomato spotted wilt virus (TSWV), or a combination thereof. Examples target sequences are shown in Table 2.

TABLE 2

| TARGET SEQUENCE ORIGIN | TARGET SEQUENCE |
|---|---|
| SARS-COV-2 | SEQ ID No. 1 |
| Respiratory Syncytial Virus (RSV) | SEQ ID No. 8 |
| H1N1 Influenza | SEQ ID No. 17 |
| Middle East Respiratory Syndrome (MERS) | SEQ ID No. 27 |
| Measles morbillivirus | SEQ ID No. 38 |
| Canine distemper virus (CDV) | SEQ ID No. 48 |
| Canine parvovirus type 2 (CPV2) | SEQ ID No. 59 |
| Canine adenovirus 1 (CAV-1) | SEQ ID No. 69 |
| Feline calcivirus (FCV) | SEQ ID No. 79 |
| Feline immunodeficiency virus (FIV) | SEQ ID No. 89 |
| Felid alphaherpesvirus 1 (FeHV-1) | SEQ ID No. 99 |
| Cucumber mosaic virus (CMV) | SEQ ID No. 110 |
| Tobacco mosaic virus (TMV) | SEQ ID No. 120 |
| Tomato spotted wilt virus (TSWV) | SEQ ID No. 130 |

Examples of primers associated with the target sequences are shown in Tables 3a and 3b. Each column of Tables 3a and 3b shows an exemplary target sequence and exemplary primer sequences associated with the target sequence in that column. For a target sequence being tested, the primer mix included in the reaction mixture may include one or more primers that are associated with the target sequence.

TABLE 3a

| | | | | | | | | |
|---|---|---|---|---|---|---|---|---|
| Target | SEQ ID | SEQ ID | SEQ ID | SEQ ID | SEQ ID | SEQ ID | SEQ ID | SEQ ID |
| Sequence | No. 1 | No. 8 | No. 17 | No. 27 | No. 38 | No. 48 | No. 59 | No. 69 |
| Forward | SEQ ID | SEQ ID | SEQ ID | SEQ ID | SEQ ID | SEQ ID | SEQ ID | SEQ ID |
| Outer (F3) | No. 2 | No. 9 | No. 18 | No. 28 | No. 39 | No. 49 | No. 60 | No. 70 |
| Reverse | SEQ ID | SEQ ID | SEQ ID | SEQ ID | SEQ ID | SEQ ID | SEQ ID | SEQ ID |
| Outer (B3) | No. 3 | No. 10 | No. 19 | No. 29 | No. 40 | No. 50 | No. 61 | No. 71 |
| Forward | SEQ ID | SEQ ID | SEQ ID | SEQ ID | SEQ ID | SEQ ID | SEQ ID | SEQ ID |
| Inner (FIP) | No. 4 | No. 11 | No. 20 | No. 30 | No. 41 | No. 51 | No. 62 | No. 72 |
| Reverse | SEQ ID | SEQ ID | SEQ ID | SEQ ID | SEQ ID | SEQ ID | SEQ ID | SEQ ID |
| Inner (BIP) | No. 5 | No. 12 | No. 21 | No. 31 | No. 42 | No. 52 | No. 63 | No. 73 |
| Forward | | SEQ ID | SEQ ID | SEQ ID | SEQ ID | SEQ ID | SEQ ID | SEQ ID |
| Inner (F2) | | No. 13 | No. 23 | No. 34 | No. 44 | No. 55 | No. 65 | No. 75 |
| Forward | | SEQ ID | SEQ ID | SEQ ID | SEQ ID | SEQ ID | SEQ ID | SEQ ID |
| Inner (F1c) | | No. 14 | No. 24 | No. 35 | No. 45 | No. 56 | No. 66 | No. 76 |
| Reverse | | SEQ ID | SEQ ID | SEQ ID | SEQ ID | SEQ ID | SEQ ID | SEQ ID |
| Inner (B2) | | No. 15 | No. 25 | No. 36 | No. 46 | No. 57 | No. 67 | No. 77 |
| Reverse | | SEQ ID | SEQ ID | SEQ ID | SEQ ID | SEQ ID | SEQ ID | SEQ ID |
| Inner (B1c) | | No. 16 | No. 26 | No. 37 | No. 47 | No. 58 | No. 68 | No. 78 |
| Forward | SEQ ID | | SEQ ID | SEQ ID | SEQ ID | SEQ ID | SEQ ID | SEQ ID |
| Loop (LF) | No. 6 | | No. 22 | No. 32 | No. 43 | No. 53 | No. 64 | No. 74 |
| Backward | SEQ ID | | | SEQ ID | | SEQ ID | | |
| Loop (LB) | No. 7 | | | No. 33 | | No. 54 | | |

TABLE 3b

| | | | | | | |
|---|---|---|---|---|---|---|
| Target | SEQ ID | SEQ ID | SEQ ID | SEQ ID | SEQ ID | SEQ ID |
| Sequence | No. 79 | No. 89 | No. 99 | No. 110 | No. 120 | No. 130 |
| Forward | SEQ ID | SEQ ID | SEQ ID | SEQ ID | SEQ ID | SEQ ID |
| Outer (F3) | No. 80 | No. 90 | No. 100 | No. 111 | No. 121 | No. 131 |
| Reverse | SEQ ID | SEQ ID | SEQ ID | SEQ ID | SEQ ID | SEQ ID |
| Outer (B3) | No. 81 | No. 91 | No. 101 | No. 112 | No. 122 | No. 132 |
| Forward | SEQ ID | SEQ ID | SEQ ID | SEQ ID | SEQ ID | SEQ ID |
| Inner (FIP) | No. 82 | No. 92 | No. 102 | No. 113 | No. 123 | No. 133 |
| Reverse | SEQ ID | SEQ ID | SEQ ID | SEQ ID | SEQ ID | SEQ ID |
| Inner (BIP) | No. 83 | No. 93 | No. 103 | No. 114 | No. 124 | No. 134 |
| Forward | SEQ ID | SEQ ID | SEQ ID | SEQ ID | SEQ ID | SEQ ID |
| Inner (F2) | No. 85 | No. 95 | No. 106 | No. 116 | No. 126 | No. 136 |
| Forward | SEQ ID | SEQ ID | SEQ ID | SEQ ID | SEQ ID | SEQ ID |
| Inner (F1c) | No. 86 | No. 96 | No. 107 | No. 117 | No. 127 | No. 137 |
| Reverse | SEQ ID | SEQ ID | SEQ ID | SEQ ID | SEQ ID | SEQ ID |
| Inner (B2) | No. 87 | No. 97 | No. 108 | No. 118 | No. 128 | No. 138 |
| Reverse | SEQ ID | SEQ ID | SEQ ID | SEQ ID | SEQ ID | SEQ ID |
| Inner (B1c) | No. 88 | No. 98 | No. 109 | No. 119 | No. 129 | No. 139 |
| Forward | | | SEQ ID | SEQ ID | | |
| Loop (LF) | | | No. 104 | No. 115 | | |
| Backward | SEQ ID | SEQ ID | SEQ ID | | SEQ ID | SEQ ID |
| Loop (LB) | No. 84 | No. 94 | No. 105 | | No. 125 | No. 135 |

Testing device 100, 300, 310 may include reaction mixtures including primers associated with one or more infectious agents that are capable of infecting the same organism. For example, testing device 100, 300, 310 may include primers associated with one or more human viruses (e.g., H1N1, SARS-COV-2, RSV, MERS, and/or measles), one or more canine viruses (e.g., CDV, CPV2, and/or CAV-1), one or more feline viruses (e.g., FCV, FIV, and/or FeHV-1), or one or more plant viruses (e.g., CMV, TMV, and/or TSWV).

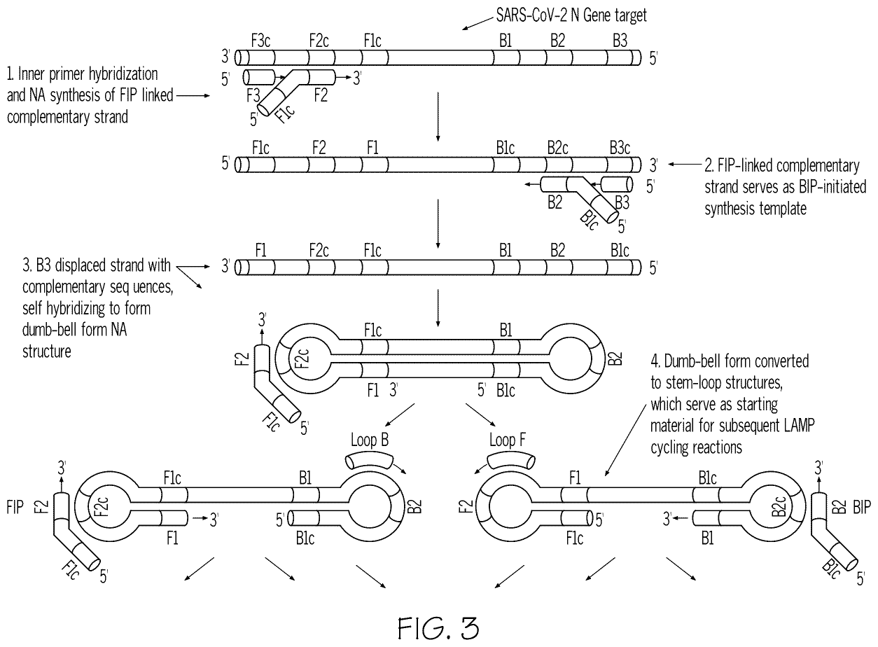

FIG. 3 includes a simplified schematic of an exemplary method of loop-mediated isothermal nucleic acid amplification (LAMP). First, inner primer hybridization and nucleic acid synthesis of FIP linked complementary strand of the target sequence is performed. Next, FIP-linked complementary strand serves as a BIP-initiated synthesis template. After that, a B3 displaced strand with complementary sequences self-hybridizes to form dumb-bell shaped nucleic acid structure. Then the dumb-bell shaped nucleic acid structure is converted to stem-loop structures, serving as a starting material for subsequent LAMP cycling reactions.

The testing device 100 may include one or more heating elements 112 configured to heat the sample reaction chambers 108 and control reaction chambers 109. Heating element 112 may function to adjust the temperature of sample reaction chambers 108 and control reaction chambers 109 to initiate a reaction (e.g., a nucleic acid amplification reaction). In some embodiments, sealing the sealing mechanism with the sample chamber 104 may trigger a circuit for the heating element 112, which is in thermal communication with the sample reaction chambers 108 and control reaction chambers 109 to initiate nucleic acid amplification reactions.

Once the sample has been distributed into sample reaction chambers 108, heating element 112 may heat the reaction mixtures in the sample reaction chambers 108 and control reaction chambers 109 to a preset temperature (e.g., a consistent temperature). The heating element may heat the reaction mixtures in the sample reaction chambers 108 and control reaction chambers 109 to a temperature of approximately 50° C. to approximately 80° C., such as, for example, approximately 60° C. to approximately 70°, approximately 60° to approximately 65° C., or approximately 65° C. Heating element 112 may heat the reaction mixtures for a heating duration. Heating duration may be approximately 5 minutes to 60 minutes, such as, for example approximately 30 minutes. Testing device 100 and heating element 112 may be configured to have different heating durations for different reaction chambers 108, 109. For example, testing device 100, 300, 310 and heating element 112 may be configured to heat a first sample reaction chamber 108 and a first control reaction chamber 109 to a first temperature for a first heating duration, and configured to heat a second sample reaction chamber 108 and a second control reaction chamber 109 to a second temperature for a second heating duration.

In some embodiments, heating element 112 may be an electrical-based heating element. Some examples of an electrical-based heating element 112 may be Peltier element, a resistive heater, the combination thereof, or any other type of electrical heater suitable for use in testing device 100. In addition or alternatively, heating element 112 may produce heat via an exothermic reaction, such as the oxidation of a metal, the crystallization of a salt, or a combination thereof. Heating element 112 may be powered by a battery, a direct current from a power source, an alternating current from a power source, or any other suitable power source for testing device 100.

Referring again to FIGS. 1A-1C, testing device 100, 300, 310 may include a circuit board 116. Circuit board 116 may be in communication with one or more probes 114 and/or a data processor 118. Circuit board 116 may receive information on the status of reaction chambers 108, 109, via the probes 114. For example, each sample reaction chamber 108 and control reaction chamber 109 may include at least a portion of a corresponding probe 114. Each probe 114 may be in communication (e.g., via contact or a connecting electrical lead) with the circuit board 116. Circuit board 116 may communicate data received from probes 114 to data processor 118. Data processor 118 may include a processor, a memory, a storage, and/or a combination thereof. The memory may be any suitable configuration of memory, such as RAM, non-volatile memory, or other suitable type of memory, and may be implemented locally or from a remote database. In some examples, the data processor 118 has executable computer readable instructions for receiving and processing the data received from each probe 114 (e.g., via circuit board 116).

The processor 118 may generate a signal based on a plurality of different inputs which may result in the processor 118 facilitating actuation of aspects of the diagnostic system. In some examples, the processor 118 may generate a first signal based on detection of the sealing mechanism being sealed against housing 102 (e.g., in response to a pressure sensor signal). In this example, in response to the detection of the first signal by the processor 118, the heating element 112 and/or the plurality of probes 114 may be activated, since the sealing mechanism has been sealed with the housing 102 indicating that the sample has moved from the sample chamber 104 to the sample reaction chambers 108. Similarly, after a designated time, the processor 118 may receive a signal from the plurality of probes 114 indicating test data from the sample reaction chambers 108 and control data from the control chambers 109, so that the processor 118 may out one or more result indicators on the display 120.

In some embodiments, probes 114 may be configured to detect a pH, a conductivity, a temperature, a viscosity, a transmittance, an absorbance, or a combination thereof, of a reaction mixture with reaction chambers 108, 109. In some examples, the probes 114 may be comprised of an ion selective glass material that is permeable to hydrogen ions. In another example, the probes 114 may be a solid-state pH sensor, such as available those commercially available from Zimmer & Peacock AS in Horten, Norway.

In some examples, data processor 118 may be configured to compare data from a probe 114 in communication with a sample reaction chamber 108 to data from a probe in communication with a control reaction chamber 109 corresponding to the sample reaction chamber 108. For example, data processor 118 may compare a first pH received from a first probe 114 in communication with a sample reaction chamber 108 to a second pH received from a second probe in communication with a control reaction chamber 109. In some examples, any change of pH greater than 0.6 point on the pH scale of the sample within the sample reaction chamber 108 more than the control reaction chamber may indicate a positive amplification. In another example, a pH threshold may be determined based on any pH shift in the sample chamber 108 greater than or equal to the difference between the average of change of pH of the control sample (e.g., pH of the control sample before nucleic acid amplification compared with the pH of the control sample after nucleic acid amplification). In some examples, the average pH change of the control sample may be gathered over multiple tests, such as averaging two or more, three or more, five or more, and even ten or more results to determine the threshold pH of the control sample. In addition or alternatively, data processor 118 may compare a first conductivity received from a first probe 114 in communication with a sample reaction chamber 108 to a second conductivity received from a second probe in communication with a control reaction chamber 109. Based on the comparison of data received from different probes 114, data processor 118 may determine the presence of one or more target sequences in the sample, and therefore, determine the presence of one or more infectious agents in the sample.

Data processor 118 may be further configured produce a readable output on display 120, which may be electronically coupled to data processor 118, for example by a circuit board 116. For example, based on the determination that one or more target sequences are in the sample, data processor 118 may induce display 120 to display, project, or otherwise indicate the presence of the target sequences in the sample. For example, display 120 may be configured to produce one or more visual representations and/or indicators, such as a text, a symbol, light, or a combination thereof, to indicate whether sample has tested positive for a particular target sequence or infectious agent. For example, display 120 may be configured to produce a first visual indicator, such as a first colored light, to indicate a positive test result, and a second visual indicator, different than the first visual indicator, such as a second colored light, to indicate a negative result. In addition or alternatively, display 120 may be configured to display first visual indicator as text indicating a positive test result (i.e. “POSITIVE” or “+”) or a second visual indicator to indicate a negative test result (i.e., “NEGATIVE” or “−”). Display 120 may be configured to display another visual indicator indicating that a test is in progress once the sealing mechanism has been engaged. For example, the third visual indicator may be a third colored light, text, or a pattern of light (e.g., a flashing light). In some embodiments, display 120 may include a screen, for example a LCD screen.

In systems of the present disclosure, testing device 100 may communicate data received from probes 114, and/or data derived from data received from probes 114, to another device of the system. For example, a component of testing device 100, 300, 310 (e.g., data processor 118) may communicate data to an external computer or other device. Examples of external computers include, but are not limited to, a personal computer, a laptop, a cell phone, a tablet, a cloud-based computer, or a combination thereof. The external computer may connect with the data processor 118 of the testing device 100, 300, 310 through a wired (e.g., USB connection; Ethernet connection) or a wireless connection (e.g., Bluetooth®; WiFi; cellular).

Advantageously, testing device 100, 300, 310, including reagents within reaction chambers 108, 109, may be stored at room temperature (e.g., approximately 20° C. to approximately 25° C.). A shelf life of testing device 100, 300, 310, when stored at room temperature, may be at least approximately 6 months.

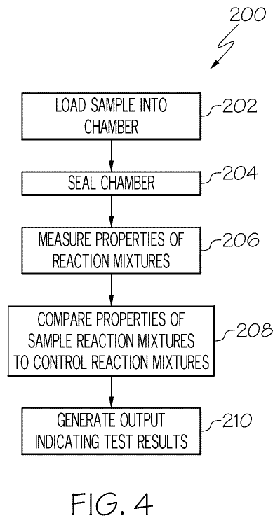

Referring to FIG. 4, a flowchart of an exemplary method 200 of using a testing device 100 is shown. Method 200 may include loading a sample into a sample chamber 104 (step 202). After the sample is loaded into the sample chamber 104, method 200 may include sealing the sample chamber 104 (step 204). Sealing the sample chamber 104 may include distributing the sample into a plurality of channels 106 and/or initiating heating of the reaction mixtures by the heating element 112. In addition or alternatively, sealing the sample chamber 104 may include preventing the sample from migrating from the sample reaction chambers 108 to the sample chamber 104.

Method 200 may include measuring properties of reaction mixtures, such as, for example, measuring properties of reaction mixtures in sample reaction chambers 108 and control reaction chambers 109 (step 206). Measuring properties may include measuring a pH, a conductivity, a temperature, a viscosity, a transmittance, an absorbance, or a combination thereof, or each reaction chamber 108, 109. Method 200 may further include comparing properties of sample reaction mixtures (i.e., mixtures within sample reaction chambers 108) and control reaction mixtures (i.e., mixtures within control reaction chambers 109) (step 208). The comparison of properties of sample reaction mixtures and control reaction mixtures may inform whether one or more target sequences and/or infectious agents are present in the sample. Method 200 may further include generating an output indicating test results (step 210). The test results may include determinations on whether one or more target sequences and/or infectious agents are present in the sample. Outputs indicating the test results may include outputs communicated via display 120 or outputs communicated via another device in communication with testing device 100.

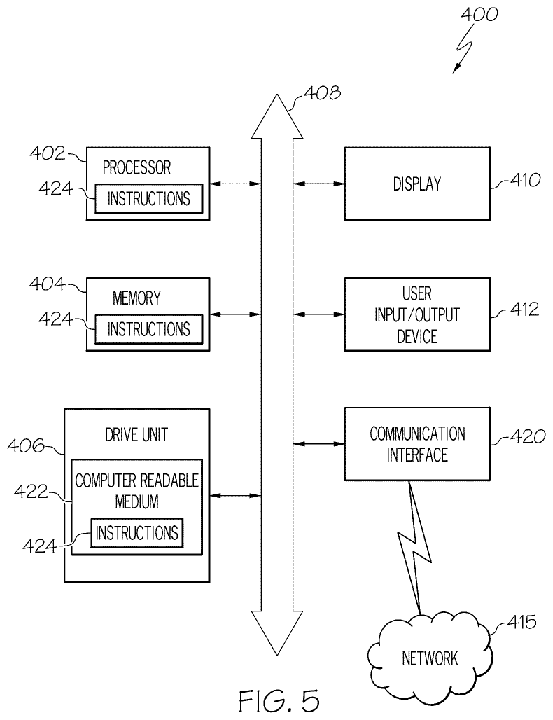

FIG. 5 is a simplified functional block diagram of a computer system 400 that may be configured as a device for executing the techniques disclosed herein, according to exemplary embodiments of the present disclosure. For example, computer system 400, or portions thereof, may be implemented as part of testing device 100 of FIG. 1A, testing device 300 of FIG. 1B, and/or testing device 310 of FIG. 1C, or may be used to perform one or more operations associated with method 200 of FIG. 4. FIG. 5 is a simplified functional block diagram of a computer system that may generate signals, analyze results, communicate with external devices and/or perform operations in accordance with embodiments of the present disclosure. In various embodiments, any of the systems (e.g., computer system 400) disclosed herein may be an assembly of hardware including, for example, a data communication interface 420 for packet data communication. The computer system 400 also may include a central processing unit (“CPU”) 402, in the form of one or more processors, for executing program instructions 424. The computer system 400 may include an internal communication bus 408, and a storage unit 406 (such as ROM, HDD, SDD, etc.) that may store data on a computer readable medium 422, although the computer system 400 may receive programming and data via network communications (e.g., over network 415). The computer system 400 may also have a memory 404 (such as RAM) storing instructions 424 for executing techniques presented herein, although the instructions 424 may be stored temporarily or permanently within other modules of computer system 400 (e.g., processor 402 and/or computer readable medium 422). The computer system 400 also may include input and output ports 412 and/or a display 410 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the disclosed subject matter may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the presently disclosed methods, devices, and systems are described with exemplary reference to transmitting data (e.g., from a component of testing device 100 to another component and/or to an external computer, as discussed herein), it should be appreciated that the presently disclosed embodiments may be applicable to any environment, such as a desktop or laptop computer, a mobile device, a wearable device, an application, or the like. Also, the presently disclosed embodiments may be applicable to any type of Internet protocol. While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

What is claimed is:

1. A diagnostic device, comprising:
a sample chamber configured to receive a sample from a patient;
a plurality of reaction chambers,
wherein the reaction chambers are configured to facilitate a loop-mediated isothermal amplification (LAMP) test;
a heating element in thermal communication with the plurality of reaction chambers;
a plurality of probes extending from each of the reaction chambers of the plurality of reaction chambers to a data processor; and
a display coupled to the data processor.

2. The diagnostic device of claim 1, wherein the display is configured to display text indicating a positive test result or a negative test result.

3. The diagnostic device of claim 1, wherein the display is configured to display a colored light indicating a positive test result.

4. The diagnostic device of claim 1, wherein each of the reaction chambers of the plurality of reaction chambers includes at least one of a reagent, a primer, or a polymerase.

5. The diagnostic device of claim 1, further comprising a one-way sealing mechanism configured to lock the sample within the sample chamber.

6. The diagnostic device of claim 1, further comprising a plurality of one-way valves disposed between each of the reaction chambers of the plurality of reaction chambers and the sample chamber.

7. The diagnostic device of claim 1, wherein the plurality of reaction chambers are at least one sample reaction chamber and at least one control chamber, the sample reaction chamber configured to receive the sample and the control chamber is configured to be isolated from the sample.

8. The diagnostic device of claim 1, wherein the heating element is configured to heat the reaction chambers to a consistent temperature for a period of 30 minutes.

9. The diagnostic device of claim 1, wherein the heating element is configured to heat the reaction chambers to a temperature in a range of 50° C. to 72° C.

10. The diagnostic device of claim 9, wherein the temperature is 65° C.

11. The diagnostic device of claim 1, wherein the LAMP test is conducted within plurality of reaction chambers.

12. The diagnostic device of claim 1, wherein the data processor is configured to compare pH data collected by the plurality of probes to control data, and generate test results based on the comparison of the pH data collected by the plurality of probes and the control data, and wherein the display is configured to generate a visual representation of the test results.

13. The diagnostic device of claim 1, wherein the data processor is configured to compare conductivity data collected by the plurality of probes to control data, and generate test results based on the comparison of the conductivity data collected by the plurality of probes and the control data, and wherein the display is configured to generate a visual representation of the test results.

14. A diagnostic device, comprising:
a sample chamber configured to receive a sample from a patient;
a plurality of reaction chambers fluidly coupled to the sample chamber,
wherein the reaction chambers are configured to facilitate a loop-mediated isothermal amplification (LAMP) test;
a heating element surrounding the plurality of reaction chambers,
wherein the heating element is configured to heat the reaction chambers to a temperature in a range of 50° C. to 72° C.;
a plurality of probes extending from each of the reaction chambers of the plurality of reaction chambers to a data processor,
wherein each of the probes of the plurality of probes is configured to provide test data to the data processor, and
wherein the data processor is configured to generate test results based on a comparison of the test data and control data; and
a display coupled to the data processor,
wherein the display is configured to generate a visual representation of the test results generated by the data processor.

15. The diagnostic device of claim 14, further comprising a one-way sealing mechanism configured to lock the sample within the sample chamber.

16. The diagnostic device of claim 15, wherein the one-way sealing mechanism is configured to push the sample from the sample chamber into the plurality of reaction chambers.

17. The diagnostic device of claim 14, wherein the plurality of reaction chambers includes at least one sample reaction chambers and at least one control reaction chamber.

18. The diagnostic device of claim 17, wherein the LAMP test is configured to test for at least influenza, SARS-COV-2, respiratory syncytial virus, Middle Eastern Respiratory Syndrome (MERS), and measles simultaneously.

19. A method of diagnosing an infectious disease, the method comprising:
loading a sample into a sample chamber of a diagnostic device;
closing the sample chamber,
wherein closing the chamber causes the sample to be pushed from the sample chamber into a plurality of reaction chambers;
conducting loop-mediated isothermal amplification (LAMP) tests within the plurality of sample chambers;
comparing test data generated by the LAMP tests to control data; and
generating an output indicating a test result based on the comparison of the test data and the control data.

20. The method of claim 19, further comprising displaying the output on a display.


SEQUENCE LISTING (139 sequences)

SEQ ID NO: 1 - DNA, 1200 bp, Severe acute respiratory syndrome coronavirus 2
  source 1..1200: mol_type = genomic DNA
gtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactc

SEQ ID NO: 2 - DNA, 19 bp, synthetic construct
  source 1..19: mol_type = other DNA
tggctactaccgaagagct

SEQ ID NO: 3 - DNA, 20 bp, synthetic construct
  source 1..20: mol_type = other DNA
tgcagcattgttagcaggat

SEQ ID NO: 4 - DNA, 41 bp, synthetic construct
  source 1..41: mol_type = other DNA
tctggcccagttcctaggtagtgacgaattcgtggtggtga

SEQ ID NO: 5 - DNA, 40 bp, synthetic construct
  source 1..40: mol_type = other DNA
agacggcatcatatgggttgcagcgggtgccaatgtgatc

SEQ ID NO: 6 - DNA, 25 bp, synthetic construct
  source 1..25: mol_type = other DNA
tggactgagatctttcattttaccg

SEQ ID NO: 7 - DNA, 24 bp, synthetic construct
  source 1..24: mol_type = other DNA
actgagggagccttgaatacacca

SEQ ID NO: 8 - DNA, 532 bp, Respiratory syncytial virus
  source 1..532: mol_type = genomic DNA
ggggcaaataagaatttgataagtaccacttaaatttaactcccttggttagagatgggcagcaattcattgagtatgataaaagttagattacaaaatttgtttgacaatgatgaagtagcattgttaaaaataacatgctatactgacaaattaatacatttaactaatgcattggctaaggcagtgatacatacaatcaaattgaatggcattgtatttgtgcatgttattacaagtagtgatatttgccctaataataatattgtagtgaaatccaatttcacaacaatgccagtgttacaaaatggaggttatatatgggaaatgatggaattaacacactgctctcaacctaatggcctaatagatgacaattgtgaaattaaattctccaaaaaactaagtgattcaacaatgaccaattatatgaatcaattatctgaattacttggatttgatcttaatccataaattataataaatatcaactagcaaatcaatgtcactaacaccattagttaatataaaa

SEQ ID NO: 9 - DNA, 21 bp, synthetic construct
  source 1..21: mol_type = other DNA
ggcattgtatttgtgcatgtt

SEQ ID NO: 10 - DNA, 23 bp, synthetic construct
  source 1..23: mol_type = other DNA
tgaatcacttagttttttggaga

SEQ ID NO: 11 - DNA, 50 bp, synthetic construct
  source 1..50: mol_type = other DNA
ctggcattgttgtgaaattggatttattacaagtagtgatatttgcccta

SEQ ID NO: 12 - DNA, 50 bp, synthetic construct
  source 1..50: mol_type = other DNA
tgggaaatgatggaattaacacactatttcacaattgtcatctattaggc

SEQ ID NO: 13 - DNA, 25 bp, synthetic construct
  source 1..25: mol_type = other DNA
attacaagtagtgatatttgcccta

SEQ ID NO: 14 - DNA, 25 bp, synthetic construct
  source 1..25: mol_type = other DNA
ctggcattgttgtgaaattggattt

SEQ ID NO: 15 - DNA, 25 bp, synthetic construct
  source 1..25: mol_type = other DNA
atttcacaattgtcatctattaggc

SEQ ID NO: 16 - DNA, 25 bp, synthetic construct
  source 1..25: mol_type = other DNA
tgggaaatgatggaattaacacact

SEQ ID NO: 17 - DNA, 1400 bp, Influenza A virus
  source 1..1400: mol_type = genomic DNA
acaaggtttctcccagtagccggcggaacaggcagtgtttatattgaagtgttgcacttaacccaagggacgtgctgggagcagatgtacactccaggaggagaagtgagaaatgatgatgttgaccaaagtttgattatcgctgctagaaacatagtaagaagagcagcagtgtcagcagacccattagcatctctcttggaaatgtgccacagcacacagattggaggagtaaggatggtggacatccttagacagaatccaactgaggaacaagccgtagacatatgcaaggcagcaatagggttgaggattagctcatctttcagttttggtgggttcactttcaaaaggacaagcggatcatcagtcaagaaagaagaagaagtgctaacgggcaacctccaaacactgaaaataagagtacatgaagggtatgaagaattcacaatggttgggagaagagcaacagctattctcagaaaggcaaccaggagattgatccagttgatagtaagcgggagagacgagcagtcaattgctgaggcaataattgtggccatggtattctcacaggaggattgcatgatcaaggcagttaggggcgatctgaactttgtcaatagggcaaaccagcgactgaaccccatgcaccaactcttgaggcatttccaaaaagatgcaaaagtgcttttccagaactggggaattgaatccatcgacaatgtgatgggaatgatcggaatactgcccgacatgaccccaagcacggagatgtcgctgagagggataagagtcagcaaaatgggagtagatgaatactccagcacggagagagtggtagtgagtattgaccgatttttaagggttagagatcaaagagggaacgtactattgtctcccgaagaagtcagtgaaacgcaaggaactgagaagttgacaataacttattcgtcatcaatgatgtgggagatcaatggccctgagtcagtgctagtcaacacttatcaatggataatcaggaactgggaaattgtgaaaattcaatggtcacaagatcccacaatgttatacaacaaaatggaatttgaaccatttcagtctcttgtccctaaggcaaccagaagccggtacagtggattcgtaaggacactgttccagcaaatgcgggatgtgcttgggacatttgacactgtccaaataataaaacttctcccctttgctgctgccccaccagaacagagtaggatgcaattttcctcattgactgtgaatgtgagaggatcagggttgaggatactggtaagaggcaattctccagtattcaattacaacaaggcaaccaaacgacttacagttcttggaaaggatgcaggtgcattgactgaagatccagatga

SEQ ID NO: 18 - DNA, 19 bp, synthetic construct
  source 1..19: mol_type = other DNA
actggggaattgaatccat

SEQ ID NO: 19 - DNA, 21 bp, synthetic construct
  source 1..21: mol_type = other DNA
caatagtacgttccctctttg

SEQ ID NO: 20 - DNA, 42 bp, synthetic construct
  source 1..42: mol_type = other DNA
atccctctcagcgacatctccacaatgtgatgggaatgatcg

SEQ ID NO: 21 - DNA, 46 bp, synthetic construct
  source 1..46: mol_type = other DNA
agagtcagcaaaatgggagtagatatctctaacccttaaaaatcgg

SEQ ID NO: 22 - DNA, 20 bp, synthetic construct
  source 1..20: mol_type = other DNA
ggggtcatgtcgggcagtat

SEQ ID NO: 23 - DNA, 21 bp, synthetic construct
  source 1..21: mol_type = other DNA
acaatgtgatgggaatgatcg

SEQ ID NO: 24 - DNA, 21 bp, synthetic construct
  source 1..21: mol_type = other DNA
atccctctcagcgacatctcc

SEQ ID NO: 25 - DNA, 22 bp, synthetic construct
  source 1..22: mol_type = other DNA
atctctaacccttaaaaatcgg

SEQ ID NO: 26 - DNA, 24 bp, synthetic construct
  source 1..24: mol_type = other DNA
agagtcagcaaaatgggagtagat

SEQ ID NO: 27 - DNA, 1995 bp, Middle East Respiratory Syndrome-related coronavirus
  source 1..1995: mol_type = genomic DNA
atgatacactcagtgtttctactgatgttcttgttaacacctacagaaagttacgttgatgtagggccagattctgttaagtctgcttgtattgaggttgatatacaacagactttctttgataaaacttggcctaggccaattgatgtttctaaggctgacggtattatataccctcaaggccgtacatattctaacataactatcacttatcaaggtctttttccctatcagggagaccatggtgatatgtatgtttactctgcaggacatgctacaggcacaactccacaaaagttgtttgtagctaactattctcaggacgtcaaacagtttgctaatgggtttgtcgtccgtataggagcagctgccaattccactggcactgttattattagcccatctaccagcgctactatacgaaaaatttaccctgcttttatgctgggttcttcagttggtaatttctcagatggtaaaatgggccgcttcttcaatcatactctagttcttttgcccgatggatgtggcactttacttagagctttttattgtattctagagcctcgctctggaaatcattgtcctgctggcaattcctatacttcttttgccacttatcacactcctgcaacagattgttctgatggcaattacaatcgtaatgccagtctgaactcttttaaggagtattttaatttacgtaactgcacctttatgtacacttataacattaccgaagatgagattttagagtggtttggcattacacaaactgctcaaggtgttcacctcttctcatctcggtatgttgatttgtacggcggcaatatgtttcaatttgccaccttgcctgtttatgatactattaagtattattctatcattcctcacagtattcgttctatccaaagtgatagaaaagcttgggctgccttctacgtatataaacttcaaccgttaactttcctgttggatttttctgttgatggttatatacgcagagctatagactgtggttttaatgatttgtcacaactccactgctcatatgaatccttcgatgttgaatctggagtttattcagtttcgtctttcgaagcaaaaccttctggctcagttgtggaacaggctgaaggtgttgaatgtgatttttcacctcttctgtctggcacacctcctcaggtttataatttcaagcgtttggtttttaccaattgcaattataatcttaccaaattgctttcacttttttctgtgaatgattttacttgtagtcaaatatctccagcagcaattgctagcaactgttattcttcactgattttggattacttttcatacccacttagtatgaaatccgatctcagtgttagttctgctggtccaatatcccagtttaattataaacagtccttttctaatcccacatgtttgattttagcgactgttcctcataaccttactactattactaagcctcttaagtacagctatattaacaagtgctctcgtcttctttctgatgatcgtactgaagtacctcagttagtgaacgctaatcaatactcaccctgtgtatccattgtcccatccactgtgtgggaagacggtgattattataggaaacaactatctccacttgaaggtggtggctggcttgttgctagtggctcaactgttgccatgactgagcaattacagatgggctttggtattacagttcaatatggtacagacaccaatagtgtttgccccaagcttgaatttgctaatgacacaaaaattgcctctcaattaggcaattgcgtggaatattccctctatggtgtttcgggccgtggtgtttttcagaattgcacagctgtaggtgttcgacagcagcgctttgtttatgatgcgtaccagaatttagttggctattattctgatgatggcaactactactgtttgcgtgcttgtgttagtgttcctgtttctgtcatctatgataaa

SEQ ID NO: 28 - DNA, 18 bp, synthetic construct
  source 1..18: mol_type = other DNA
cagggagaccatggtgat

SEQ ID NO: 29 - DNA, 19 bp, synthetic construct
  source 1..19: mol_type = other DNA
agaacccagcataaaagca

SEQ ID NO: 30 - DNA, 47 bp, synthetic construct
  source 1..47: mol_type = other DNA
cgtcctgagaatagttagctacaaagtatgtttactctgcaggacat

SEQ ID NO: 31 - DNA, 43 bp, synthetic construct
  source 1..43: mol_type = other DNA
taggagcagctgccaattccgtaaatttttcgtatagtagcgc

SEQ ID NO: 32 - DNA, 24 bp, synthetic construct
  source 1..24: mol_type = other DNA
acttttgtggagttgtgcctgtag

SEQ ID NO: 33 - DNA, 24 bp, synthetic construct
  source 1..24: mol_type = other DNA
actggcactgttattattagccca

SEQ ID NO: 34 - DNA, 22 bp, synthetic construct
  source 1..22: mol_type = other DNA
gtatgtttactctgcaggacat

SEQ ID NO: 35 - DNA, 25 bp, synthetic construct
  source 1..25: mol_type = other DNA
cgtcctgagaatagttagctacaaa

SEQ ID NO: 36 - DNA, 23 bp, synthetic construct
  source 1..23: mol_type = other DNA
gtaaatttttcgtatagtagcgc

SEQ ID NO: 37 - DNA, 20 bp, synthetic construct
  source 1..20: mol_type = other DNA
taggagcagctgccaattcc

SEQ ID NO: 38 - DNA, 1689 bp, Measles morbillivirus
  source 1..1689: mol_type = genomic DNA
aggattcaagatcctattatcggggacaagagcaggattagggtaatccgagatggccacacttttaaagagcttagctttgttcaagagaagcaaggacaaaccacccattacatcaggatccggtggagccatcagaggaatcaaacacattattatagtaccaatccctggggattcctcaattaccactcgatctagacttctggaccgattggttaggttgatcggaaacccggatgtaagcgggcccaaactaacaggggcactaataggtatattgtccttatttgtggagtctccaggccaattgattcagaggatcaccgatgacccagacgttagcataaggctattagaagttgtccagagtgaccaatcacaatccggccttaccttcgcgtcaagaggtaccaacatggaggatgaggcagaccaatacttttcacatgatgatccaagcagtggtgatcaatccaggttcggatggttcgagaacaaggaaatctcagatattgaagtgcaagatcccgagggattcaacatgattctgggtactatcctagctcaaatttgggtcttgctcgcaaaggcggttactgccccagacacggcagctgattcggagctaagaaggtggataaagtacacccaacaaagaagggtagtcggtgaatttagattggagagaaaatggttggatgtggtgaggaataggattgctgaggacctctccttacgtcgattcatggtcgctctaatcctggacatcaagagaacacctgggaacaaacccaggattgctgaaatgatatgtgacattgacacatatatcgtagaagcaggattagccagttttattctgactattaagtttgggatagaaaccatgtatcctgctcttggactgcatgaatttgctggtgagttatccacacttgagtccctgatgaatctttatcaacaaatgggggaaactgcaccttacatggtaattctggagaactcaattcagaacaaattcagtgcaggatcgtaccctctgctctggagctatgccatgggagtaggagtggaacttgaaaactccatgggaggtttgaactttggccgatcttactttgatccggcatattttagattaggacaagagatggttaggagatcagctggaaaggtcagttccacactggcatctgaactcggtatcacagccgaggatgcaaggcttgtttcagagattgcaatgcatactactgaggacaggaccagcagagcagttggacccaggcaagcccaagtctcatttttacacagtgatcaaagtgagaatgagctaccgggattggggggcaaggaagatagaagggtcaaacagagtcgaggggaaaccagggagaactccagagaaaccgggcccggcagagcaagtgatgcgagagctgcccatctcccaaccagcacacccccagacattgacactgcatcggagtacagccaagaaccacaagacagtcgaaggtcagctgacgccctgctcaagctgcaagccatggcagggatcctggaagaacaaggctcagacacggacacccctagagtgtacaacgatagagatcttctagactaggtacaagagaccggggaccagaacaacatccgcctaccctccatcattgttataaaaaa

SEQ ID NO: 39 - DNA, 19 bp, synthetic construct
  source 1..19: mol_type = other DNA
cccccagacattgacactg

SEQ ID NO: 40 - DNA, 19 bp, synthetic construct
  source 1..19: mol_type = other DNA
gggtaggcggatgttgttc

SEQ ID NO: 41 - DNA, 39 bp, synthetic construct
  source 1..39: mol_type = other DNA
tggcttgcagcttgagcaggcggagtacagccaagaacc

SEQ ID NO: 42 - DNA, 40 bp, synthetic construct
  source 1..40: mol_type = other DNA
aggctcagacacggacaccccccggtctcttgtacctagt

SEQ ID NO: 43 - DNA, 19 bp, synthetic construct
  source 1..19: mol_type = other DNA
gcgtcagctgaccttcgac

SEQ ID NO: 44 - DNA, 19 bp, synthetic construct
  source 1..19: mol_type = other DNA
cggagtacagccaagaacc

SEQ ID NO: 45 - DNA, 20 bp, synthetic construct
  source 1..20: mol_type = other DNA
tggcttgcagcttgagcagg

SEQ ID NO: 46 - DNA, 20 bp, synthetic construct
  source 1..20: mol_type = other DNA
cccggtctcttgtacctagt

SEQ ID NO: 47 - DNA, 20 bp, synthetic construct
  source 1..20: mol_type = other DNA
aggctcagacacggacaccc

SEQ ID NO: 48 - DNA, 1572 bp, Canine distemper virus
  source 1..1572: mol_type = genomic DNA
atggctagccttcttaagagcctcacattattcaagaggactcgggaccaacccccacttgcctcgggctccggaggagcaataagagggataaagcatgtcattatagtcctaatcccgggtgactcaagcattgttacaagatcacgactgttggatagacttgttagattggtcggtgatccggaaatcaacgggcctaaattaactgggattttaatcagtatcctctccttgttcgtggaatcccctggacaattgattcagaggatcatagacgaccctgatgtaagcatcaaattagtagaggtaataccaagcatcaactctgtttgcggtcttacatttgcatccagaggagcaagtttggattctgaggcagatgagttctttaaaattacagacgaagggtcgaaagctcagggacaattaggctggttggagaataaggatattgtagacatagaagttgatgatgctgagcaattcaatatattgctagcttccatcttggcccaaatttggatcctgctcgctaaagcagtgaccgctcccgacactgcagccgattcggagatgagaaggtggattaagtatacccaacagagacgtgtggtcggggaattcagaatgaacaaaatatggcttgatattgttagaaacagaattgctgaggacttatctttgaggcggttcatggtggcactcatcttggatatcaaacgatccccagggaacaagcctagaattgctgaaatgatttgtgatatcgataactacattgtggaagctggattagctagtttcatcttaactatcaaatttggcattgaaactatgtatccggctcttgggttgcatgagttctccggggagttaacaactattgaatcccttatgatgctatatcaacagatgggtgaaacagcaccgtacatggttattctggaaaattctgttcagaacaaatttagtgcagggtcctacccattgctctggagttatgctatgggagttggtgttgaacttgaaaactccatgggaggattaaatttcggtagatcctactttgatccagcttatttcaggctcgggcaagaaatggttagaagatctgccggcaaagtaagctctgcacttgccgccgagcttggcatcaccaacgaagaggctcagctagtgtcagaaatagcatccaagacaacggaggaccggacgattcgcgctgctggtcccaagcaatctcaaatcacttttctgcactcagaaagatccgaagtcaccaatcaacagcccccagccattaacaagaggtccgaaaaccaaggaggagacaaataccccatccatttcggtgatgaacggtttccagggtataccccagatgtcaacagctccgaatggagtgaatcacgctatgatacccaaactatccaagatgatgggaacgacgatgaccggaagtcgatggaagcaatcgccaagatgagaatgcttaccaagatgctcagtcaacctgggaccagcgaagagagctcacctgtctataatgatagagagctactcaattaa

SEQ ID NO: 49 - DNA, 18 bp, synthetic construct
  source 1..18: mol_type = other DNA
agcttggcatcaccaacg

SEQ ID NO: 50 - DNA, 20 bp, synthetic construct
  source 1..20: mol_type = other DNA
gggtataccctggaaaccgt

SEQ ID NO: 51 - DNA, 42 bp, synthetic construct
  source 1..42: mol_type = other DNA
gagattgcttgggaccagcagcaagaggctcagctagtgtca

SEQ ID NO: 52 - DNA, 42 bp, synthetic construct
  source 1..42: mol_type = other DNA
aagtcaccaatcaacagccccctggggtatttgtctcctcct

SEQ ID NO: 53 - DNA, 19 bp, synthetic construct
  source 1..19: mol_type = other DNA
atcgtccggtcctccgttg

SEQ ID NO: 54 - DNA, 23 bp, synthetic construct
  source 1..23: mol_type = other DNA
agccattaacaagaggtccgaaa

SEQ ID NO: 55 - DNA, 20 bp, synthetic construct
  source 1..20: mol_type = other DNA
aagaggctcagctagtgtca

SEQ ID NO: 56 - DNA, 22 bp, synthetic construct
  source 1..22: mol_type = other DNA
gagattgcttgggaccagcagc

SEQ ID NO: 57 - DNA, 20 bp, synthetic construct
  source 1..20: mol_type = other DNA
tggggtatttgtctcctcct

SEQ ID NO: 58 - DNA, 22 bp, synthetic construct
  source 1..22: mol_type = other DNA
aagtcaccaatcaacagccccc

SEQ ID NO: 59 - DNA, 1330 bp, Canine parvovirus 2
  source 1..1330: mol_type = genomic DNA
atggcacctccggcaaagagagccaggagaggacttgtgcctccaggttataaatatcttgggcctgggaacagtcttgaccaaggagaaccaactaacccttctgacgccgctgcaaaagaacacgacgaagcttacgctgcttatcttcgctctggtaaaaacccatacttatatttctcgccagcagatcaacgctttatagatcaaactaaggacgctaaagattggggggggaaaataggacattatttttttagagctaaaaaggcaattgctccagtattaactgatacaccagatcatccatcaacatcaagaccaacaaaaccaactaaaagaagtaaaccaccacctcatattttcatcaatcttgcaaaaaaaaaaaaagccggtgcaggacaagtaaaaagagacaatcttgcaccaatgagtgatggagcagttcaaccagacggtggtcagcctgctgtcagaaatgaaagagctacaggatctgggaacgggtctggaggcgggggtggtggtggttctgggggtgtggggatttctacgggtactttcaataatcagacggaatttaaatttttggaaaacggatgggtggaaatcacagcaaactcaagcagacttgtacatttaaatatgccagaaagtgaaaattatagaagagtggttgtaaataatttggataaaactgcagttaacggaaacatggctttagatgatacccatgcacaaattgtaacaccttggtcattggttgatgcaaatgcttggggagtttggtttaatccaggagattggcaactaattgttaatactatgagtgagttgcatttagttagttttgaacaagaaatttttaatgttgttttaaagactgtttcagaatctgctactcagccaccaactaaagtttataataatgatttaactgcatcattgatggttgcattagatagtaataatactatgccatttactccagcagctatgagatctgagacattgggtttttatccatggaaaccaaccataccaactccatggagatattattttcaatgggatagaacattaataccatctcatactggaactagtggcacaccaacaaatatataccatggtacagatccagatgatgttcaattttatactattgaaaattctgtgccagtacacttactaagaacaggtgatgaatttgctacaggaacatttttttttgattgtaaaccatgtagactaacacatacatggcaaacaaatagagcattgggcttaccaccatttctaaattctttgcctcaagctgaaggaggta

SEQ ID NO: 60 - DNA, 18 bp, synthetic construct
  source 1..18: mol_type = other DNA
tttctcgccagcagatca

SEQ ID NO: 61 - DNA, 18 bp, synthetic construct
  source 1..18: mol_type = other DNA
tttacttgtcctgcaccg

SEQ ID NO: 62 - DNA, 49 bp, synthetic construct
  source 1..49: mol_type = other DNA
gagcaattgcctttttagctctaaatttatagatcaaactaaggacgct

SEQ ID NO: 63 - DNA, 49 bp, synthetic construct
  source 1..49: mol_type = other DNA
atacaccagatcatccatcaacatcgcaagattgatgaaaatatgaggt

SEQ ID NO: 64 - DNA, 24 bp, synthetic construct
  source 1..24: mol_type = other DNA
aataatgtcctattttcccccccc

SEQ ID NO: 65 - DNA, 24 bp, synthetic construct
  source 1..24: mol_type = other DNA
tttatagatcaaactaaggacgct

SEQ ID NO: 66 - DNA, 25 bp, synthetic construct
  source 1..25: mol_type = other DNA
gagcaattgcctttttagctctaaa

SEQ ID NO: 67 - DNA, 24 bp, synthetic construct
  source 1..24: mol_type = other DNA
gcaagattgatgaaaatatgaggt

SEQ ID NO: 68 - DNA, 25 bp, synthetic construct
  source 1..25: mol_type = other DNA
atacaccagatcatccatcaacatc

SEQ ID NO: 69 - DNA, 1827 bp, Canine mastadenovirus A
  source 1..1827: mol_type = genomic DNA
atgtctctgaacgcgctggactgtgcccgcctaaccggacaaaccccatacaccgttgaggtgttcaggcctatcagaaacatttttaaccgggtccgagaatataccagagcttccaccacctctgtagggctggcctggatgtccaaatacatataccaataccatcgcctcatgcttatgaatttgagccctcgagaacctgccacagaagggtggccgctttttttgtacccaccgccccacctcctggtgggttaccagtacctagttagaacgtgtaatgactatgtttttgacaccaggtcctacagcagactaaaatatacagagatacacctgcccttgcaacaaaaactaaattggacggtgatggccaactgctcatacaccattaacactggggcttaccataggttcattgactttgagaactttgaagagaccctggctcaggttcagcaggcggttctggcagagcgcgtggtagccgacctggctctcattagacccatgaggggctatggcactacgaatatggcaggtgacagacaggttcccgtagaggggcttctgcaagatcactacaaaaacctgtcccagtgtcaaaaccatgcatggggcttggcagaccgcatgcgaatccagaatgcgggcaacaaagacatagtgattctaaccaccatccgcaagcttaagacagctttttttaactttttagtcagccctcgaaacccacacaccatactaagcttaccttgtgactgtttgtggctcgacgcctttatgcagaaattcactgatccgactctgtcacagtttcagactattcagtctttaccaagtcaatctgtaacaaagagcataattagcgctctgagcctacccgggcccgcgccctgcaccccactatcgggaggagccttcgagcttaggcccagggaaaatggacgtgcagttacagaagaaatgagacgcagacggggagaaatgatagagagatttatagacaggcttccaatgcgcagacgcagacgcagggctcccccgccgcctccgatgtctgaggagctctctgaacccgaagtagaggcctttcctcctgcctcccctcctcgccgctcctttgaagaagaagtccgagacactatagtagaagctatccgcctcctgcaagaggaacttacagtttctgcccgcaatgaacagttttttaattttgcaattaacttttatgaagttatacagcgcttggagatgctgggcaacattaatgagctcaccattcgccgttgggtcatgtatttttttgtggccgagcacgtcgccaccacacttaactacctgcatcacaaccttagactgtacccaccctgctcccgctgggttgatcttgagctagcccaggtggtcatgcgggctagggatcatgaaggacaggtagtttatagtagagtgtggaatgaaatgggagaaaatgctttcagccagcttatggctcgcgtgtctggtgatctggctgctacagtagaaagggccgggctaggagagctagaagaagaagaaatggaacaatttatggcagacattgcctatcatgaaaatagtggggatgtgagcgaaattctaaggcaggtggccattaatgacacagaggttgattctatggaactttcttttaggttcaaagtcacgggaccagtggtcttttcacagaaccgccaaattcaatcaatcaacaggagagtagtggccctagcctcccagctcaggatgcaacacaggcctctgccagctcagcacgagcaggtgcaactcccgccataa

SEQ ID NO: 70 - DNA, 19 bp, synthetic construct
  source 1..19: mol_type = other DNA
ctggcctggatgtccaaat

SEQ ID NO: 71 - DNA, 19 bp, synthetic construct
  source 1..19: mol_type = other DNA
agggcaggtgtatctctgt

SEQ ID NO: 72 - DNA, 42 bp, synthetic construct
  source 1..42: mol_type = other DNA
cggtgggtacaaaaaaagcggccaataccatcgcctcatgct

SEQ ID NO: 73 - DNA, 40 bp, synthetic construct
  source 1..40: mol_type = other DNA
acctcctggtgggttaccagttgctgtaggacctggtgtc

SEQ ID NO: 74 - DNA, 24 bp, synthetic construct
  source 1..24: mol_type = other DNA
ggttctcgagggctcaaattcata

SEQ ID NO: 75 - DNA, 20 bp, synthetic construct
  source 1..20: mol_type = other DNA
caataccatcgcctcatgct

SEQ ID NO: 76 - DNA, 22 bp, synthetic construct
  source 1..22: mol_type = other DNA
cggtgggtacaaaaaaagcggc

SEQ ID NO: 77 - DNA, 19 bp, synthetic construct
  source 1..19: mol_type = other DNA
tgctgtaggacctggtgtc

SEQ ID NO: 78 - DNA, 21 bp, synthetic construct
  source 1..21: mol_type = other DNA
acctcctggtgggttaccagt

SEQ ID NO: 79 - DNA, 1750 bp, Feline calcivirus
  source 1..1750: mol_type = genomic DNA
atgtctcaaactctgagcttcgtgcttaaaactcacagtgtccgaaaggactttgtgcactctgtcaagttaacacttgcacggaggcgcgatcttcagtatatttataacaagctctcacgcactatacgtgctgaggcttgcccttcttgtgctagttacgacgtatgtcctaactgcacctctggtgacgtcccggatgacgggtcttcgacaatgtcgattccatcatgggaggatgtcacaaagtcttcaacctactctctcctgctctctgaggacacctccgacgagttatgccctgaggacttggttaatgtggctgctcatatccgtaaggcgctatccactcagtcccacccggctaatgctgaaatgtgcaaagaacagctcactttcttattagtcatggctgaggcgatgctgccccaacgatcccgagcgtcaatcccactgcaccaacaacacacggctgcacggttggaatggagggagaaattcttctctaaacctcttgactttctccttgaaagagttggtgtgtcaaaagatattctccaaactactgcgatttggaaaattatcttggaaaaagcatgctattgtaaatcctatggagagcagtggttcactgctgccaaacaaaagttaagggaaatgaaaaactttgagagtgatacgctaaaacctcttattggtggatttatagatggtctacggttcttgaccgtggacaacccaaacccgatgggtttcctcccaaaactcatagggcttgtaaaacccctgaatttggcgatgattattgataatcatgaaaacacaatatctggctggatcatcacattaaccgcaataatggagctatacaacatcaccgaatgcaccatagatattataacatcagtcattacggctttctatgataaaattggcaaggcaaccaaattttacagttgtgttaaggcgctgttcactggatttagatctgaggacgtagcgaattccttttggtacatggcagcagcgattctatgttacctgatcactgggttaatcccaaacaacggcagattttcaaagataaaagcttgcctggccggagcgacaactcttgtatcaggtatagttgccacacaaaagctagctgcaatgttcgcaacatggaactctgagtccattgttaatgagttgtcagcaagaactgttgccctatcagagctaaacaatccaacaacaacgtctgacacggattcggtagaacgactgctagaattggctaagatcttgcatgaggagatcaagattcatactctaaaccccatcatgcaatcatacaatccaatcttgagaaatttaatgtcaaccttggacggtgttataacatcatgcaacaagaggaaagctattgctcggaagagacaggtgccagtttgttacatattaactggcccacctggatgtgggaaaacaaccgcagctcaagcattagctaagaaattgtctgatcaagagccgtcggtaattaaccttgatgttgatcatcatgacacatatactggaaatgaggtatgtatcattgatgagtttgattcgtctgacaaggtagactatgcaaattttgtgattgggatggtaaactctgctcctatggtgttaaattgtgacatgcttgagaacaagggaaagctcttcacctcaaagtatataattatgacttccaactctgaaactccagtgaaaccctctt

SEQ ID NO: 80 - DNA, 18 bp, synthetic construct
  source 1..18: mol_type = other DNA
ggagagcagtggttcact

SEQ ID NO: 81 - DNA, 19 bp, synthetic construct
  source 1..19: mol_type = other DNA
tccagccagatattgtgtt

SEQ ID NO: 82 - DNA, 49 bp, synthetic construct
  source 1..49: mol_type = other DNA
ccgtagaccatctataaatccaccaggaaatgaaaaactttgagagtga

SEQ ID NO: 83 - DNA, 40 bp, synthetic construct
  source 1..40: mol_type = other DNA
ttgaccgtggacaacccaaacaatcatcgccaaattcagg

SEQ ID NO: 84 - DNA, 22 bp, synthetic construct
  source 1..22: mol_type = other DNA
cgatgggtttcctcccaaaact

SEQ ID NO: 85 - DNA, 24 bp, synthetic construct
  source 1..24: mol_type = other DNA
ggaaatgaaaaactttgagagtga

SEQ ID NO: 86 - DNA, 25 bp, synthetic construct
  source 1..25: mol_type = other DNA
ccgtagaccatctataaatccacca

SEQ ID NO: 87 - DNA, 19 bp, synthetic construct
  source 1..19: mol_type = other DNA
aatcatcgccaaattcagg

SEQ ID NO: 88 - DNA, 21 bp, synthetic construct
  source 1..21: mol_type = other DNA
ttgaccgtggacaacccaaac

SEQ ID NO: 89 - DNA, 1509 bp, Feline immunodeficiency virus
  source 1..1509: mol_type = genomic DNA
atggggaatgagcagggtaaagaggtaaaggctgcagtcaagagatgcaaagaggtagctgtaggtccggggagtaagagcaaaagatatggagaagggaatataagatgggccatcagaatggctaatgtaactacaggacgagaccctggtgaattgccagtgaatatagacagcataagaacattagtgtgtgatttggaggagagaagagaaaaatatggttctaacaaggaaatagatgctgctatagcaactttaaaagtattaggaattgtaggtattttgtatatgaaagcttccacaacagagtccgcttgcaatttgtggaagattatgggattaaattctagaccaacagaaaagggaccaggatcagatgtagaagaaccacctaatgcttggcaagctaaagtaaatcaattaaaaagggaaggattaggattgagagaacctcaagaattagtaaaagaatatcctattcaagttctcaatggagtaccacaatatgtaccattaaatccaagaatggtggccatttttatggaaaaagcaagagaaggcttgggaacagaggaagtactgcttcggtttactgcattttcagcagatttaacccctactgatatggctactattttaatgtccgctccagggtgtgcttcagataaggaaataatagatacaaaattaaaggaattaactagtgagtatgaaagaacacatccttcagatggtccaagaccattaccatattttacagctagagaaatgatgggattggatttgacacaagatcaacaggcagcacctcattttcatcctgctcggatacagtgtagggcttggtatttagaggctctacaacatttaatgaaggtaaaagctaaaagtcctaaagcagtgcaaatgaaacaaggacctaaagaagattattcacaatttatagatagattatatgcacaaattgatcaagaacaaaatagtccagaagtaaagctatatttaaaacaatctttgagtttagctaatgctaaccctgaatgtaaaagggctatgtctcatttaaaaccagaaagcccattagaagaaaaattaagagcatgtcaggaaatagggaccacaccctataaaatgaatatgttggcacaagctttgcagaacaatgggtgtaatcaagttatgcaagcaaatgtgagaccaaaaggatcacaacaaggcaatagaagaccaggtcaatcttttaaatgctttaattgtggaaaaccaggacatttatctagacaatgtagagctcctagaaagtgtaatacttgtggaaaagcagggcatatttctacagattgtcggcaaatgaaaagaaaacaacagggaaactggcagcaggggagagctgctgcccctgtcaaacaagtgcagcaatttcagactgcagtgccaatcagtctgcaacagactgcacctgtgcagccatcagctccaccaatggaatccctattggaaatgtga

SEQ ID NO: 90 - DNA, 20 bp, synthetic construct
  source 1..20: mol_type = other DNA
gccagtgaatatagacagca

SEQ ID NO: 91 - DNA, 18 bp, synthetic construct
  source 1..18: mol_type = other DNA
atctgatcctggtccctt

SEQ ID NO: 92 - DNA, 49 bp, synthetic construct
  source 1..49: mol_type = other DNA
agcatctatttccttgttagaaccataagaacattagtgtgtgatttgg

SEQ ID NO: 93 - DNA, 48 bp, synthetic construct
  source 1..48: mol_type = other DNA
atatgaaagcttccacaacagagtgttggtctagaatttaatcccata

SEQ ID NO: 94 - DNA, 22 bp, synthetic construct
  source 1..22: mol_type = other DNA
ccgcttgcaatttgtggaagat

SEQ ID NO: 95 - DNA, 24 bp, synthetic construct
  source 1..24: mol_type = other DNA
taagaacattagtgtgtgatttgg

SEQ ID NO: 96 - DNA, 25 bp, synthetic construct
  source 1..25: mol_type = other DNA
agcatctatttccttgttagaacca

SEQ ID NO: 97 - DNA, 24 bp, synthetic construct
  source 1..24: mol_type = other DNA
gttggtctagaatttaatcccata

SEQ ID NO: 98 - DNA, 24 bp, synthetic construct
  source 1..24: mol_type = other DNA
atatgaaagcttccacaacagagt

SEQ ID NO: 99 - DNA, 1605 bp, Felid alphaherpesvirus 1
  source 1..1605: mol_type = genomic DNA
atgagacgatataggatgggacgcggaatctaccttctctatatctgtctgttatatacatatctccagtttggtacttcgtcgacaaccgcggtcagtattgaaaatagtgataatagtactgcggagatgttatcatctaccagcatgtccgctaccaccccgatatcccagccaacatctccattcactactccaactagaagatctacaaatatagctacaagttcgagtaccacccaggcatcccagccaacatctacattaactactctaactagaagctcgacaactatagctacaagtccgagtaccacccaggcagccacattcataggatcatctaccgattccaataccactttactcaaaacaacaaaaaaaccaaagcgtaaaaagaataagaataacggggccagatttaaattagattgtggatataagggggttatctacagaccgtattttagccctcttcagctaaactgtactctacccacagaacctcatattaccaaccctattgacttcgagatctggtttaaaccacgcaccagatttggggattttcttggggataaagaagacttcgtagggaatcatacccgcaccagcatattactatttagcagccgtaatgggagtgttaattccatggatcttggggacgcgacactcgggatcctacaatctaggataccagattacacattatataatattcccatacaacataccgaagcgatgtcattgggaatcaaatctgtggaatctgccacgtccggtgtttatacatggcgggtctatggtggagatggactaaataaaacagtgctaggacaggtaaatgtatctgtagtggcatatcaccccccgagcgtaaatcttacaccacgcgccagtctatttaataagacctttgaggcggtatgtgcagtggcgaattacttccccccgcgatccacgaaactaacatggtatcttgacgggaagccaatagaaaggcaatacatttcagatacggcaagtgtatggatagatggactcatcaccagaagttctgtgttggctattccgacaactgaaacagattccgagaaaccagatatacgatgtgatttggaatggcatgaaagtcctgtgtcctataagagattcacgaaaagtgtagccccggacgtctattacccacctactgtgtctgttaccttcgctgatacacgggctatatgtgatgttaaatgtgtaccacgggacgggatatccttgatgtggaaaattggtaactaccatctaccaaaagcaatgagtgctgatatactgatcacaggtccgtgtatagaacgtccaggtttggtcaacattcagagtatgtgtgatatatcagaaacggatggacccgtgagttatacctgtcagaccatcggatacccaccaattctaccgggattttacgacacacaagtctacgacgcgtcccctgaaatcgtcagtgaatcaatgttggttagtgtcgttgctgtaatactaggagctgttctcatcacagtctttatctttattacggcattatgtttatattattctcatccccggcgattataa

SEQ ID NO: 100 - DNA, 23 bp, synthetic construct
  source 1..23: mol_type = other DNA
tctaggataccagattacacatt

SEQ ID NO: 101 - DNA, 20 bp, synthetic construct
  source 1..20: mol_type = other DNA
cgtggtgtaagatttacgct

SEQ ID NO: 102 - DNA, 41 bp, synthetic construct
  source 1..41: mol_type = other DNA
catgtataaacaccggacgtggatacaacataccgaagcga

SEQ ID NO: 103 - DNA, 40 bp, synthetic construct
  source 1..40: mol_type = other DNA
ggtctatggtggagatggactggggtgatatgccactaca

SEQ ID NO: 104 - DNA, 25 bp, synthetic construct
  source 1..25: mol_type = other DNA
tccacagatttgattcccaatgaca

SEQ ID NO: 105 - DNA, 25 bp, synthetic construct
  source 1..25: mol_type = other DNA
aaacagtgctaggacaggtaaatgt

SEQ ID NO: 106 - DNA, 19 bp, synthetic construct
  source 1..19: mol_type = other DNA
atacaacataccgaagcga

SEQ ID NO: 107 - DNA, 22 bp, synthetic construct
  source 1..22: mol_type = other DNA
catgtataaacaccggacgtgg

SEQ ID NO: 108 - DNA, 19 bp, synthetic construct
  source 1..19: mol_type = other DNA
ggggtgatatgccactaca

SEQ ID NO: 109 - DNA, 21 bp, synthetic construct
  source 1..21: mol_type = other DNA
ggtctatggtggagatggact

SEQ ID NO: 110 - DNA, 657 bp, Cucumber mosaic virus
  source 1..657: mol_type = genomic DNA
atggacaaatctgaatcaaccagtgctggtcgtaaccgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtagacctgggtacacgttcacatctattaccctaaagccaccaaaaatagaccgtgggtcttattacggtaaaaggttgttactacctgattcagtcacggaatatgataagaagcttgtttcgcgcattcaaattcgagttaatcctttgccgaaatttgattctaccgtgtgggtgacagtccgtaaagttcctgcctcctcggacttatccgttgccgccatctctgctatgttcgcggacggagcctcaccggtactggtttatcagtatgccgcatctggagtccaagccaacaacaaactgttgtatgatctttcggcgatgcgcgctgatataggtgacatgagaaagtacgccgtcctcgtgtattcaaaagacgatgcgctcgagacggacgagctagtacttcatgttgacatcgagcaccaacgcattcccacatctggagtgctcccagtctga

SEQ ID NO: 111 - DNA, 20 bp, synthetic construct
  source 1..20: mol_type = other DNA
tgattctaccgtgtgggtga

SEQ ID NO: 112 - DNA, 20 bp, synthetic construct
  source 1..20: mol_type = other DNA
cggcgtactttctcatgtca

SEQ ID NO: 113 - DNA, 40 bp, synthetic construct
  source 1..40: mol_type = other DNA
ctccgtccgcgaacatagcacagtccgtaaagttcctgcc

SEQ ID NO: 114 - DNA, 40 bp, synthetic construct
  source 1..40: mol_type = other DNA
cagtatgccgcatctggagtcccgcatcgccgaaagatca

SEQ ID NO: 115 - DNA, 20 bp, synthetic construct
  source 1..20: mol_type = other DNA
agatggcggcaacggataag

SEQ ID NO: 116 - DNA, 20 bp, synthetic construct
  source 1..20: mol_type = other DNA
cagtccgtaaagttcctgcc

SEQ ID NO: 117 - DNA, 20 bp, synthetic construct
  source 1..20: mol_type = other DNA
ctccgtccgcgaacatagca

SEQ ID NO: 118 - DNA, 18 bp, synthetic construct
  source 1..18: mol_type = other DNA
cgcatcgccgaaagatca

SEQ ID NO: 119 - DNA, 22 bp, synthetic construct
  source 1..22: mol_type = other DNA
cagtatgccgcatctggagtcc

SEQ ID NO: 120 - DNA, 480 bp, Tobacco mosaic virus
  source 1..480: mol_type = genomic DNA
atgtcttacagtatcactactccatctcagttcgtgttcttgtcatcagcgtgggccgacccaatagagttaattaatttatgtactaatgccttaggaaatcagtttcaaacacaacaagctcgaactgtcgttcaaagacaattcagtgaggtgtggaaaccttcaccacaagtaactgttaggttccctgacagtgactttaaggtgtacaggtacaatgcggtattagacccgctagtcacagcactgttaggtgcattcgacactagaaatagaataatagaagttgaaaatcaggcgaaccccacgactgccgaaacgttagatgctactcgtagagtagacgacgcaacggtggccataaggagcgcgataaataatttaatagtagaattgatcagaggaaccggatcttataatcggagctctttcgagagctcttctggtttggtttggacctctggtcctgcaacttga

SEQ ID NO: 121 - DNA, 18 bp, synthetic construct
  source 1..18: mol_type = other DNA
taggttccctgacagtga

SEQ ID NO: 122 - DNA, 21 bp, synthetic construct
  source 1..21: mol_type = other DNA
aaattatttatcgcgctcctt

SEQ ID NO: 123 - DNA, 43 bp, synthetic construct
  source 1..43: mol_type = other DNA
gcacctaacagtgctgtgacctttaaggtgtacaggtacaatg

SEQ ID NO: 124 - DNA, 45 bp, synthetic construct
  source 1..45: mol_type = other DNA
tagaagttgaaaatcaggcgaacctctactctacgagtagcatct

SEQ ID NO: 125 - DNA, 18 bp, synthetic construct
  source 1..18: mol_type = other DNA
ccacgactgccgaaacgt

SEQ ID NO: 126 - DNA, 23 bp, synthetic construct
  source 1..23: mol_type = other DNA
ctttaaggtgtacaggtacaatg

SEQ ID NO: 127 - DNA, 20 bp, synthetic construct
  source 1..20: mol_type = other DNA
gcacctaacagtgctgtgac

SEQ ID NO: 128 - DNA, 21 bp, synthetic construct
  source 1..21: mol_type = other DNA
tctactctacgagtagcatct

SEQ ID NO: 129 - DNA, 24 bp, synthetic construct
  source 1..24: mol_type = other DNA
tagaagttgaaaatcaggcgaacc

SEQ ID NO: 130 - DNA, 777 bp, Tomato spotted wilt orthotospovirus
  source 1..777: mol_type = genomic DNA
atgtctaaggttaagctcactaaggaaagcattgttgctttgttgacacaaggcaaagaccttgagtttgaggaagatcagaatctggtagcattcaacttcaagactttttgtctggaaaacatcgaccagatcaagaagatgagcgttatttcatgtctgacattcctaaagaatcgtcagagcataatgaaggttattaagcaaagcgattttacttttggtaaaattaccataaagaaaacttcagacaggattggaggcactgacatgaccttcagaaggcttgatagcttgatcagggtcaggcttgttgaagaaactgggaattctgagaatctcaatactatcaaatctaagattgcttcccatcctttgattcaagcctatggattacctctcgatgatgcaaagtctgtgagacttgccataatgctgggaggtagcttacctcttattgcttcagttgatagctttgagatgatcagtgttgtcttggctatatatcaggatgcaaaatacaaggacctcgggatcgacccaaagaagtatgacaccaaggaagccttaggaaaagtttgcactgtgctgaaaagcaaagcatttgaaatgaatgaagatcaggtgaagaaggggaaagagtatgctgctatacttagctccagcaatcctaatgctaaagggagtgttgctatggaacattacagtgaaactcttaacaagttctatgaaatgttcggggttaaaaagcaggcaaaactcgcagaacttgcttga

SEQ ID NO: 131 - DNA, 22 bp, synthetic construct
  source 1..22: mol_type = other DNA
ctttgattcaagcctatggatt

SEQ ID NO: 132 - DNA, 18 bp, synthetic construct
  source 1..18: mol_type = other DNA
ttcagcacagtgcaaact

SEQ ID NO: 133 - DNA, 43 bp, synthetic construct
  source 1..43: mol_type = other DNA
gcaataagaggtaagctacctccccctctcgatgatgcaaagt

SEQ ID NO: 134 - DNA, 45 bp, synthetic construct
  source 1..45: mol_type = other DNA
atgatcagtgttgtcttggctatatttccttggtgtcatacttct

SEQ ID NO: 135 - DNA, 24 bp, synthetic construct
  source 1..24: mol_type = other DNA
gatgcaaaatacaaggacctcggg

SEQ ID NO: 136 - DNA, 19 bp, synthetic construct
  source 1..19: mol_type = other DNA
cctctcgatgatgcaaagt

SEQ ID NO: 137 - DNA, 24 bp, synthetic construct
  source 1..24: mol_type = other DNA
gcaataagaggtaagctacctccc

SEQ ID NO: 138 - DNA, 20 bp, synthetic construct
  source 1..20: mol_type = other DNA
ttccttggtgtcatacttct

SEQ ID NO: 139 - DNA, 25 bp, synthetic construct
  source 1..25: mol_type = other DNA
atgatcagtgttgtcttggctatat